(19) United States
(12) Patent Application Publication
(10) Pub. No.: US 2015/0290015 A1
(43) Pub. Date: Oct. 15, 2015

(54) Ankle Foot Orthosis Using Shape Memory Alloys for Addressing Drop Foot

(71) Applicant: The University of Toledo, Toledo, OH (US)

(72) Inventors: Mohammad H. Elahinia, Toledo, OH (US); Morteza GorzinMataee, Troy, MI (US); Masood Taheri Andani, Toledo, OH (US)

(21) Appl. No.: 14/682,198

(22) Filed: Apr. 9, 2015

Related U.S. Application Data
(60) Provisional application No. 61/977,130, filed on Apr. 9, 2014.

(51) Int. Cl. A61F 5/01

(52) U.S. Cl. CPC A61F 5/0127; A61F 2005/0132

(57) ABSTRACT

An ankle foot orthosis (AFO) is provided to address the drop foot irregularity. In two introduced embodiments of the device, a shape memory alloy (SMA) component is loaded during powered plantarflexion. The SMA would then enable the AFO to lift the foot during the dorsiflexion, when the foot drops. The AFO offers a compact and lightweight structure, however, while still providing the patient the desired lift in during dorsiflexion.

### STATEMENT REGARDING FEDERALLY SPONSORED RESEARCH

This invention was not made with any federally sponsored research or development support.

### TECHNICAL FIELD AND INDUSTRIAL APPLICABILITY OF THE INVENTION

The present invention relates a solution for treating people with drop foot.

### CROSS REFERENCE TO RELATED APPLICATIONS

This application claims the benefit of U.S. provisional patent application Ser. No. 61/977,130 filed Apr. 9, 2014.

### BACKGROUND OF THE INVENTION

Drop foot is a neuromuscular disorder described by the inability of the patient to dorsiflex (raise up) the front portion of their foot. Drop foot may be caused by various reasons including stroke, diabetes, multiple sclerosis, etc. The insufficiency is physically characterized by weakness or paralysis of the muscles around the ankle so that the patient cannot dorsiflex, but is still able to plantarflex (push down) the foot. Drop foot results in the forefoot slapping the ground at heel strike and dragging the toe during the swing phase. Affected people tend to have a labored, unsafe gait and suffer from fatigue which further reduces their speed and efficiency. At mid swing, toe drag prevents proper limb advancement and increases the risk of tripping. Without treatment, drop foot can cause severe injuries in the affected limbs and other limbs which are required to compensate for the deficiency.

Orthotics, functional electrical simulation (FES), physical therapy and surgery are the most common treatments offered for drop foot. Each method has specific advantages and disadvantages and is prescribed for the patient depending on his/her health condition, symptoms and requirements. FES has shown some promise as a permanent assistance device, but the technology must be customized to the individual using trial-and-error methods and qualitative measurements. Although both physical therapy and surgery have shown some biomechanical benefits, disadvantages preclude them as acceptable treatments for all patients.

An ankle foot orthosis (AFO) on the other hand is a rehabilitative mechanical device that supports and aligns the ankle and foot to correct drop foot. The AFO also suppresses spastic and overpowering ankle and foot muscles, assists weak and paralyzed muscles of the ensemble, prevents escalating deformities, and improves overall function for the patient. Conventional AFOs are passive plastic braces which prevent the drop foot by restricting the ankle movement during the entire gait cycle. Although these light orthosis prevent drop foot, walking is still difficult with them because they do not provide the required torsional stiffness of an ankle during normal gait. These AFOs cause too much resistance to plantarflexion, inhibiting ankle motion throughout the loading response. They also lead to disuse atrophy of the ankle flexor muscles by completely restricting the ankle movement. Another common complaint among AFO users is that they are so uncomfortable that the user often forgoes the AFO. Further, many users require modification to their footwear to accommodate the bulky AFOs currently available.

Based on input received from of patients and clinicians, there is a need to AFOs with a low weight and compact structure allow the patients to wear them on daily basis. A functional device should provide sufficient motion and stiffness in the sagittal plane of the movement required for normalizing the gait. Such as AFO should allow for wearing regular shoes and could have a single hinge for a minimalist profile. Carbon fiber AFOs with a single-sided flexible joint, offer a new less obtrusive style of AFO. Although these devices are lightweight and durable, walking stability and adjustment of compliance are two main issue to be considered.

This patent discloses embodiments of AFOs utilizing shape memory alloys to address the condition of drop foot using a more compact design, while not restricting the motion during the rest of the gait. These designs may be completely passive to eliminate peripherals related to energy transfer and control of the device. Instead, the device works on the basis of timely energy storage and release using the superelastic behavior of SMA materials. Among all designs, the common feature is simplicity of the design using complex and flexible behavior of shape memory alloys, resolving issues of weight, space, appearance and tethered operation.

### SUMMARY OF THE INVENTION

Described herein is an ankle foot orthosis device utilizing superelastic shape memory alloys to provide the force required to lift up the foot of a patient with a drop foot disorder in place of the muscles.

Shape memory alloys (SMA) are a group of smart materials that can effectively change their shape and provide actuation by restoring their memorized geometry. An example of a SMA is Nitinol, a metal alloy of nickel and titanium. The reversible mechanism behind shape memory alloy actuation is a solid-state phase transformation that takes place in response to variation of temperature and stress. The distinct thermo-mechanical behavior of SMAs is the result of a transformation from an austenite (parent) phase to a martensite (product) phase and vice versa. These alloys have very high energy density; therefore, actuators that implement these alloys are compact and lightweight SMA actuators are an effective way to reduce weight and to minimize the complexity of various systems. Biocompatibility and elastic properties close to body tissues (such as bone and tendon) are among the other reasons why SMA is used for this application.

A novel design of an articulated passive AFO using a single-sided superelastic SMA hinge is presented in this invention. It is demonstrated that the single-sided SMA hinge could provide the required motion to inhibit drop foot, and prevent unwanted deflection which results in gait instability and hypermobility.

While the present invention is directed to an AFO, the superelastic SMA hinge could be used in any orthotic where mobility and stability are desired. Possible applications include knee, elbow, or should braces.

The preferred embodiment of the present invention utilizes a single-sided SMA hinge, however, should additional lift be desired it is envisioned that a second SMA hinge would be added to aid in lifting the patients foot.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

FIG. 1 is a schematic illustration of the biomechanics of an ankle during a walking cycle.

FIG. 2 is a side view illustration of the present invention.

FIG. 3 is a perspective view of an AFO with a single-sided SMA hinge and traditional brace.

FIG. 4 is a side view of FIG. 3

FIG. 5 is a front view of the AFO with a single-sided SMA hinge and modified brace.

FIG. 6 is a perspective view of FIG. 5

FIG. 7 is a perspective view of the single-sided SMA hinge jointed to the foot and calf braces.

FIG. 8 is a perspective view of the single-sided SMA hinge detailing the pinned connection to the braces.

FIG. 9 is a perspective view of the SMA hinge.

FIG. 10 is a graph of the stiffness for a one-sided SMA hinge from numerical study to mimic ankle stiffness in swing phase

FIG. 11 is a graph of 3D deflections at the most critical loading phase of the gait for the one-sided SMA hinge

FIG. 12 is a perspective view of the single-sided SMA hinge AFO with an adjustable hinge

FIG. 13 is a graph showing the stiffness profiles from numerical study through structural adjustment of the single-sided SMA hinge, mimicking ankle stiffness in various gait speeds during the swing phase

### DETAILED DESCRIPTION

Superelasticity is the featured ability of some materials to recover large amount of deformation without any residual strain. Shape memory alloys (SMAs) exhibit this property at specific temperatures due to a solid-solid phase transformation which occurs in these materials under mechanical loading.

While any method of fabrication may be used to produce the SMA hinge, the inventors relied on Additive Manufacturing as generally described in the following publicaitons: HABERLAND, Christoph, Additive Manufacturing of Shape Memory Devices and Pseudoelastic Components. SMASIS2013-3070, and HABERLAND, Christoph, Visions, Concepts and Strategies for Smart Nitinol Actuators and Complex Nitinol Structures Produced by Additive Manufacturing. SMASIS2013-3072.

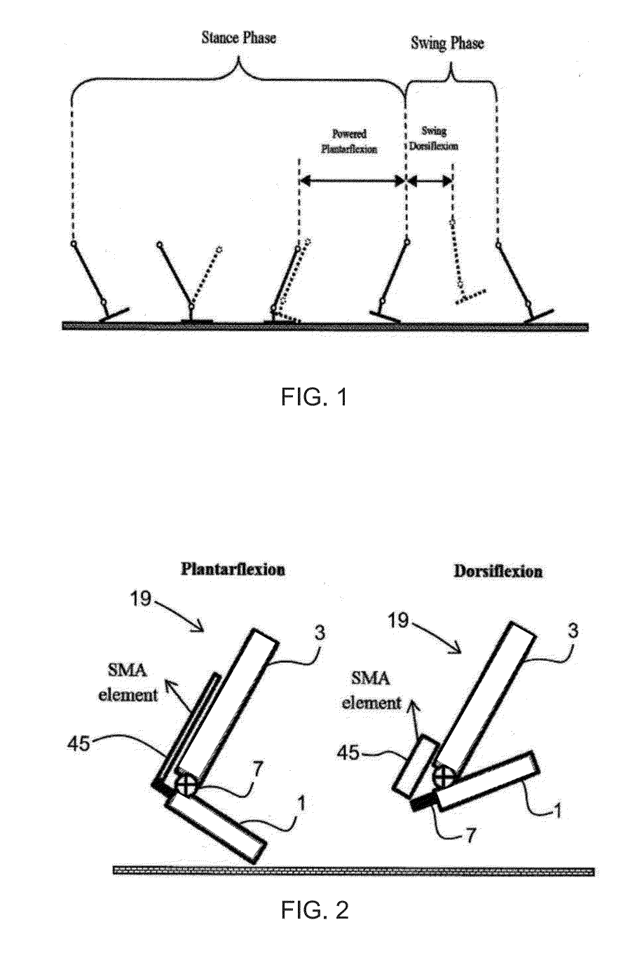

As shown in FIG. 1, during powered plantarflexion, an internal stress is induced in SMA which causes the material to be loaded. When the foot leaves the ground during the swing phase, the stress is no longer applied and the SMA is free to recover, enabling dorsiflexion (unloading). The initial condition is taken at maximum controlled dorsiflexion in order to have the maximum stress induced during powered plantarflexion. FIG. 2 is a general depiction of a passive SMA AFO 19 which constraints the motion of an ankle during walking. The passive SMA AFO 19 consists of a calf brace 3, a foot brace 1, a hinge 7, and a superelastic SMA element 45. The SMA element 45 stores the mechanical energy of the ankle during the powered plantarflexion portion of the stance phase when the patient has the ability to move his/her ankle. The passive SMA AFO 19 will then restore this energy during the swing dorsiflexion when the patient is unable to raise his/her foot by lifting the foot brace 1. The main objectives in developing the SMA based AFO are to reduce the number of moving parts and to reduce the manufacturing cost of the actuator. Moreover, the proposed design is simple, fast and lightweight. It does not inhibit the natural motion of the ankle and it does not need any complex controlling method.

The SMA AFO 19 is designed to be energized during the powered plantarflexion (in stance phase) and then raise the foot in the dorsiflexion portion of swing phase as depicted in FIG. 1. Along with energizing the superelastic element to assist the ankle in swing, the also SMA element inhibits sudden deceleration of the foot shortly after heel strike to prevent foot slap due to weak muscle dorsiflexor.

To improve portability, durability and conformability and make the fitting process easier for clinicians, a single-sided SMA hinge is designed and developed for an AFO. The single-sided SMA hinge design is a key way to reduce the profile of the brace while lowering the weight of the orthosis.

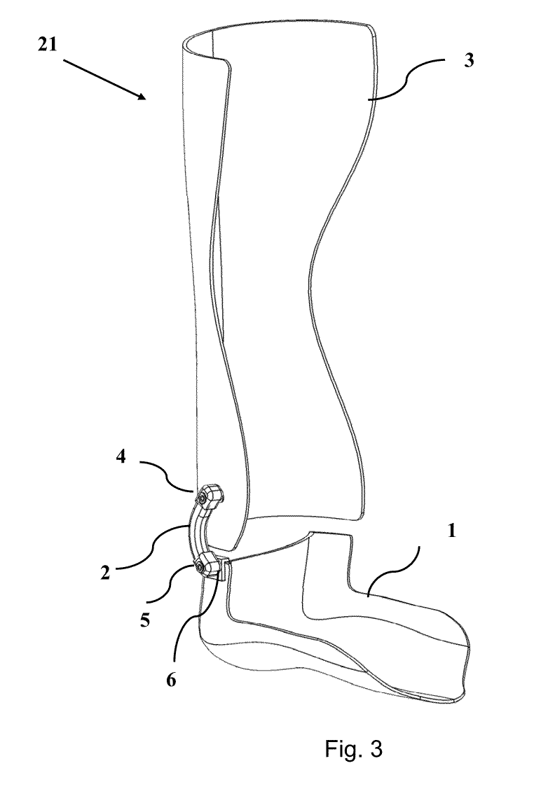

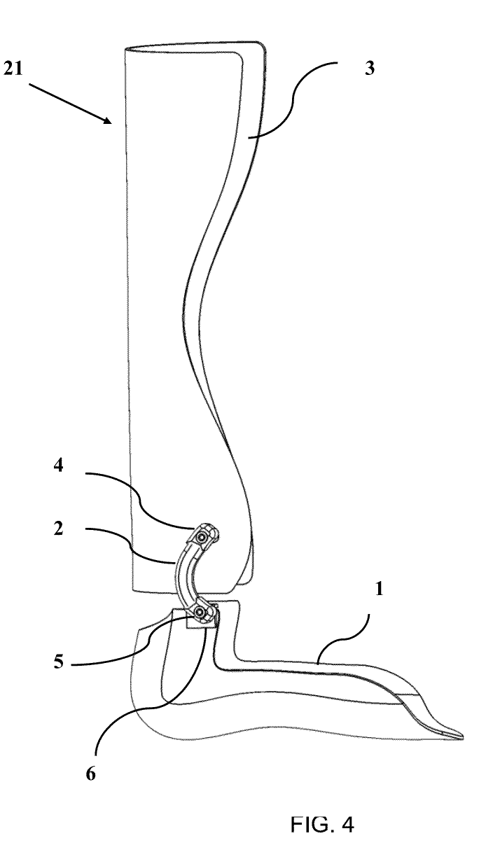

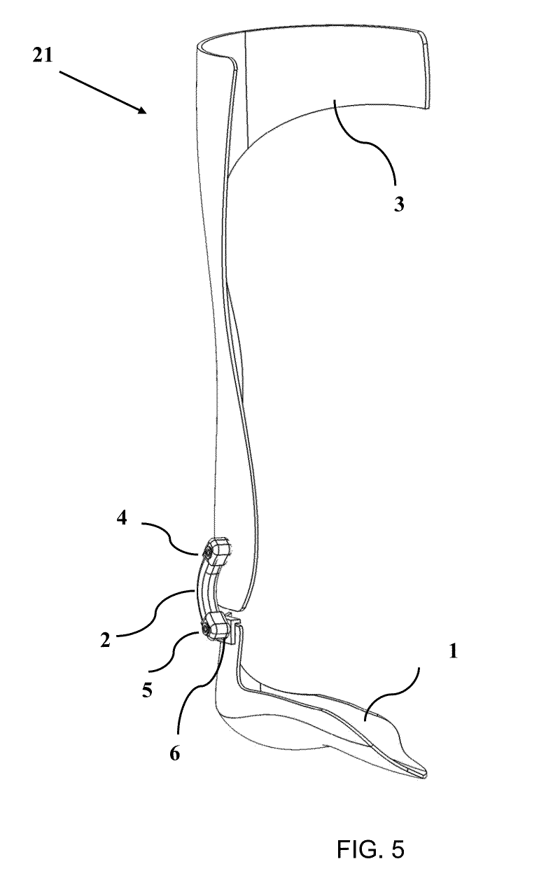

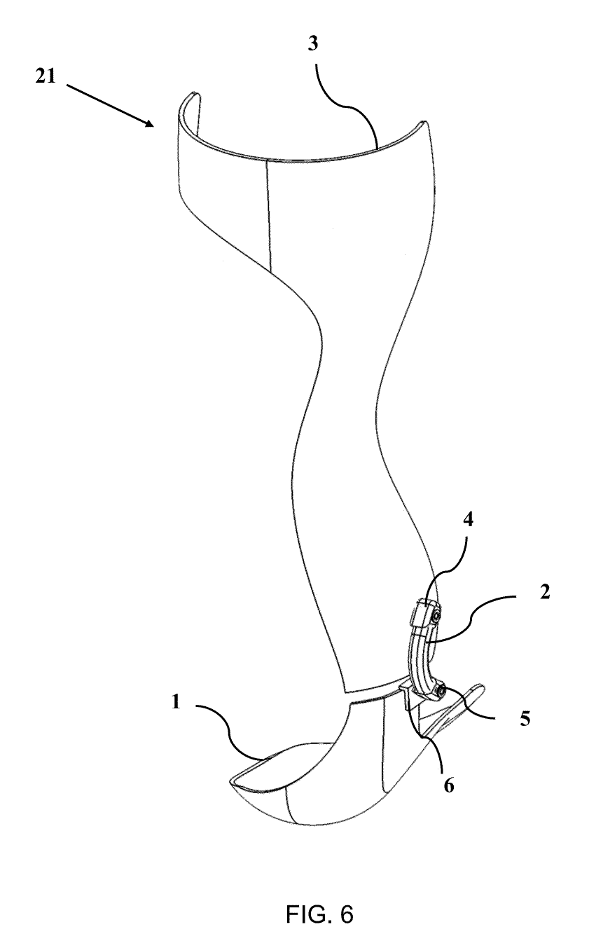

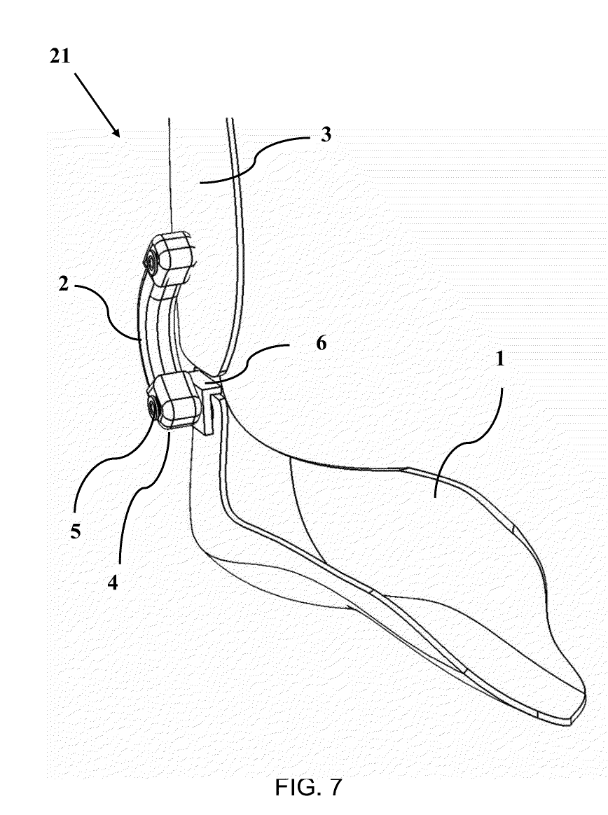

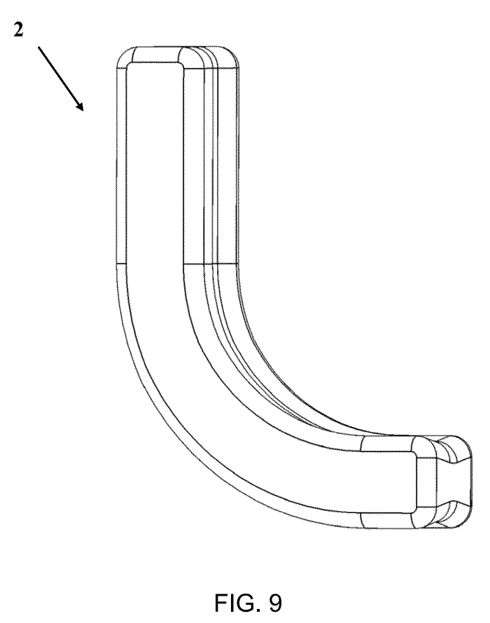

Referring now to FIGS. 3-9, the preferred embodiment is shown. An AFO 21 is shown having a foot brace 1 and a calf brace 3, which are used to hold the foot and ankle during the walking motion. The foot brace 1 is connected to the calf brace 3 on a single side by an SMA hinge 2. SMA is a metal alloy that returns to a known shape after a load is removed. The single-sided SMA hinge 2 is designed so the known shape results in dorsiflexion. During plantarflexion, the single-sided SMA hinge 2 is loaded with potential energy by deforming the SMA from its known or resting shape as the patient steps on the ground. This potential energy is converted to kinetic energy when the load is removed from the single-sided SMA hinge 2 during the swing phase. This kinetic energy causes the single-sided SMA hinge 2 to return to its known shape, which causes the foot brace 1 to swing upward resulting in dorsiflexion and the correction of the patients drop foot.

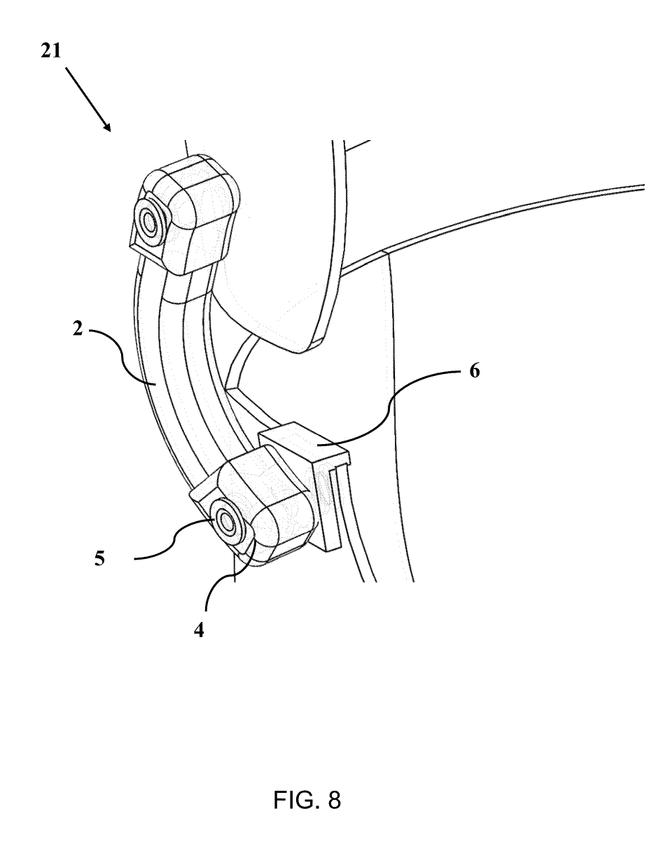

The single-sided SMA hinge 2 connects the foot brace 1 to the calf brace 3 and supports the ankle in approximate lower extremity of tibia close to the medial malleolus. This compact structure is facilely fitted in a patient's shoes. The hinge end supports 4 are connected to the brace with a pin 5 or molded within the brace profile. A pinned connection is shown with greater detail in FIGS. 7-8.

Analytical studies show that the single-sided SMA hinge 2 would provide the required motion of the ankle in the sagittal plane and prevents deflections in other directions. By considering the parallel pattern of the AFO 21 connected to the foot, the same rotation profile of the ankle is applied to the hinge element to achieve the desired moment and stiffness of the ankle in the swing. From the range of motion for a healthy and drop foot, the profile of the ankle rotation is achieved and divided to the loading and unloading modes in four different events including: loading response, mid and terminal stance, pre-swing and swing. This preliminary loading condition defines the required input in the plane of motion.

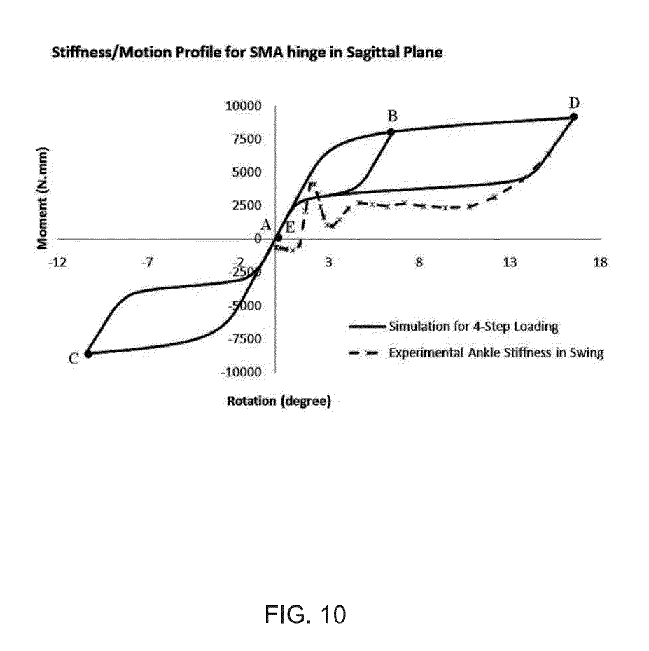

The simulation result for the single-sided SMA hinge 2 are presented in FIG. 10. The single-sided SMA hinge 2 is loaded by ankle plantarflexion during loading response (from A to B). The single-sided SMA hinge 2 is then unloaded during mid and terminal stance when the ankle dorsiflexes and decreases the moment to a negative value (at point C). During the large plantarflexion loading, the single-sided SMA hinge 2 moment reaches its maximum value (at point D), and the second hysteresis loop is formed. This happens at pre-swing when the single-sided SMA hinge is loading and preparing to recover the foot. Finally, the load is recovered in the swing and the profile returns to its starting point (from D to E). This represents the stiffness profile of the single-sided SMA hinge 2 for the entire gait, which in three hysteresis loops are formed. The most desired path is to follow the nonlinear ankle profile in swing indicated by the dash line. This profile is the main requirement of motion in sagittal plane.

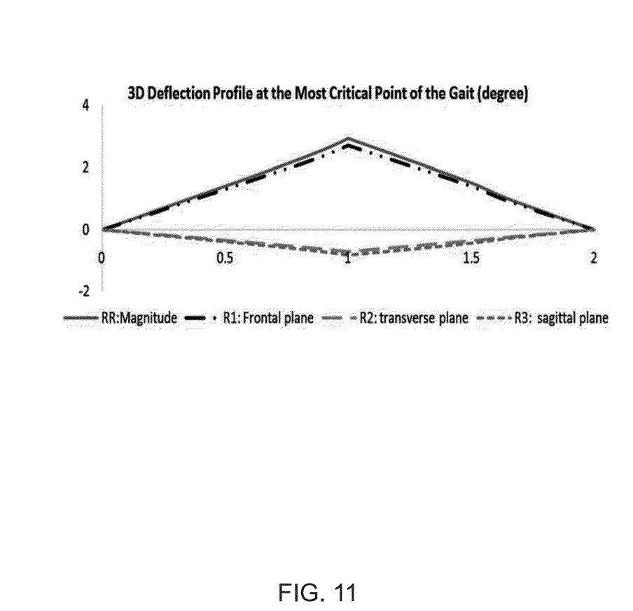

Furthermore, investigating critical loads in 3D during walking demonstrates the single-sided SMA hinge 2 with optimum dimensions would prevent unwanted motion in the transverse and frontal plane and secure walking stability. Based on the 3D gait analysis, ground reaction parameters including three force components along the three axes and distance variations of center of pressure of the foot (position of applied load) in the transverse plane are found for the whole cycle. In order to exert loading to the single-sided SMA hinge 2, the ground reaction forces are transferred to the position of the single-sided SMA hinge 2 which produce two components of moment in the frontal and transverse planes. Critical points of the gait are recognized according to the diagrams of the transferred ground reaction forces and the corresponding resultant moments at the hinge location. A FEA carried out to evaluate performance of the device by controlling the deflection and strain level. FIG. 11, illustrates the deflections of the single-sided SMA hinge 2 for the highest level of the resultant 3D loads of the gait from simulations. The results show that the deflection in the most critical point of the gait is lower than 4 degrees, therefore is negligible to induce lateral instability in movement.

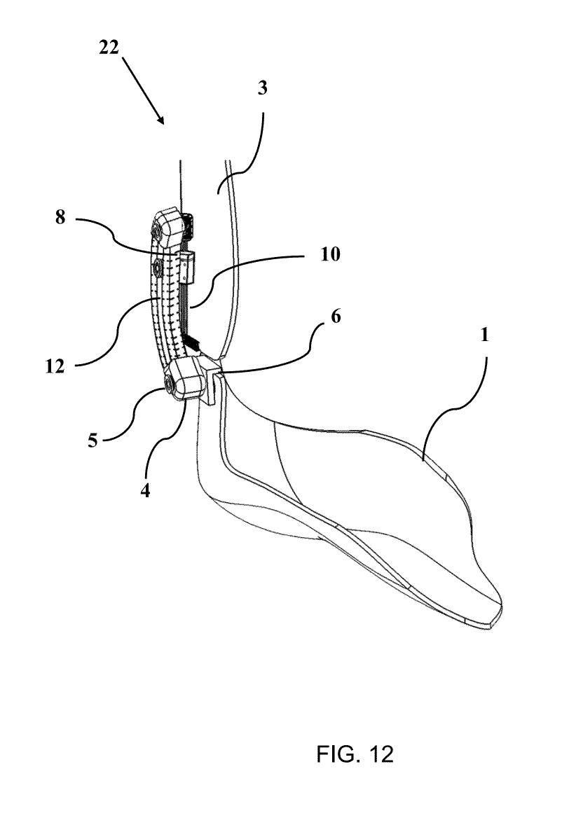

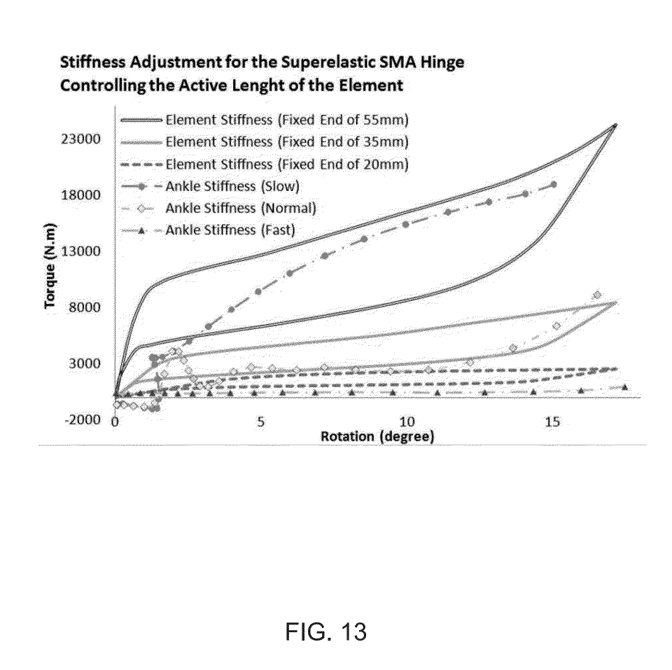

A comprehensive gait analysis demonstrate that loads applied to an AFO can be affected by movement patterns. Speed variations during walking significantly change the stiffness profile of the ankle. It is therefore desirable to develop AFOs that provide stiffness adaptation in walking. FIG. 12, displays an adjustable single-sided SMA hinge 12 for an AFO 22. In this embodiment, the stiffness of the device is controlled by a structural adjustment of the single-sided SMA hinge 12. According to a beam bending equation, bending stiffness is determined by the material modulus, moment of inertia and length of the beam. Implementing this concept herein, modulating active length of the single-sided SMA hinge 12 produces variable stiffness behavior due to the phase transformation that happens in the SMA element structure. The active length of the single-sided SMA hinge 12 is determined by the position of a slider 8 joined to one end of the single-sided SMA hinge and adjusted along a vertical motion guide 10 in the calf brace 3. The other end of the single-sided SMA hinge 12 is fixed to the foot brace 1 by a support 4 and pin 5 connected to a bracket 6.

Investigation performed from FEA revealed that the lower stiffness of the single-sided SMA hinge 12 could cover fast gait speeds occurring within the higher percentile of the single-sided SMA hinge 12 length, and that higher stiffness curves cover slow gait speeds occurring within the lower percentile of single-sided SMA hinge 12 length. From stiffness profile of the ankle in three various walking speeds of slow, normal and fast, three different lengths from optimized dimensions of the single-sided SMA hinge 12 are detected. Simulation result is shown in FIG. 11, compared to the real stiffness profiles of the ankle. To follow the ankle stiffness in slow walking, the arc length of the single-sided SMA hinge 12 is fixed at the end of 55 mm, so that the single-sided SMA hinge exhibits stiff behavior. In order to mimic the stiffness profile of normal walking, it is fixed to a length of 35 mm. Finally, for fast speed, the single-sided SMA hinge 12 is fixed at 20 mm in order to show a soft behavior.

As a preliminary evaluation of clinical efficacy, a prototype of the device as shown in FIG. 3 is fabricated and tested on a drop foot subject. In order to fit in a shoe, any kind of direct connection to the foot has been avoided. This also results in a more convenient device. Furthermore, for the cosmetic and convenient purposes the device is designed to push the heel portion of the foot brace 1 during the dorsifexion instead of pulling the front part of the foot.

To demonstrate the improvements achieved with the new devices, two tests are performed: without an AFO, and with the single-sided SMA hinge AFO 21. The collected data reveals that the SMA AFO can significantly improve the ability of the patient to raise his foot during the dorsiflexion that is the ideal case and happens for a healthy foot.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. An ankle foot orthosis having a calf brace and a foot support, the foot support being moveably connected to the calf brace, comprising:
a shape memory alloy (SMA) element attached to the calf brace and the foot support, the SMA element moveably connecting the foot support to the calf brace and the SMA element configured to deform when force is applied during a powered plantarflexion phase of a walk cycle when the foot support is moved with respect to the calf brace and to recover when force is removed during a dorsiflexion phase of a walk cycle, treating foot gait pathology, the SMA element disposed to lift the foot support during the dorsiflexion phase of a walk cycle.

2. The apparatus of claim 1 wherein the SMA material is nitinol.

3. The apparatus of claim 1 wherein the SMA element is a hinge that is secured to one end of the calf brace and one end of the foot support.

4. The apparatus of claim 3 wherein the hinge is positioned on one side of the calf brace and the foot support.

5. The apparatus of claim 3 wherein the hinge is adjustable to vary the force supplied by the hinge to lift the foot.

6. The apparatus of claim 5 wherein the hinge is adjusted to control movement between the calf brace and the foot support.

7. The apparatus of claim 5 wherein a guide is positioned on the calf brace and the end of the hinge that is secured to the calf brace is moveably secured to the guide.

8. The apparatus of claim 8 wherein a slider is secured to the hinge and the slider is moveably positioned on the guide; the position of the slider on the guide controls the active length of the hinge and the force supplied by the hinge to the foot support.

9. A method of operating an ankle foot orthosis containing a shape memory alloy (SMA) comprising:
storing mechanical energy in the SMA during the powered plantarflexion phase of a walk cycle by forcing the SMA into a deformed position; and
releasing the stored mechanical energy in the SMA during the dorsiflexion in swing phase of a walk cycle by removing the force and returning SMA to a resting position; and Lifting the foot by release of the stored mechanical energy to provide toe clearance and reducing drag during a dorsiflexion phase of a walk cycle.

10. A shape memory alloy (SMA) element for use in orthotics, comprising:
an orthotic having a first support, moveably connected to a second support, the first and second supports disposed to define a range of motion, a hinge shaped SMA element operatively connecting the first and second supports, wherein the SMA element is configured to deform when force is applied and to recover when force is removed, wherein the hinge shaped SMA element controls the range of motion of the first and second supports.
